(19) United States
(12) Patent Application Publication
(10) Pub. No.: US 2009/0156837 A1
(43) Pub. Date: Jun. 18, 2009

(54) ISOLATION AND RECOVERY OF SIMVASTATIN IN LACTONE FORM OR IN THE FORM OF AN ACID SALT FROM THE HARVESTED FERMENTATION BROTH

(75) Inventors: Dinesh Shantilal PATEL, Mumbai (IN); Sachin Dinesh Patel, Mumbai (IN); Shashikant Prabhudas Kurani, Mumbai (IN); Tapas Barui, Mumbai (IN); Rajneesh Anand, Mambai (IN); T. Damodar Reddy, Mumbai (IN)

Correspondence Address: MERCHANT & GOULD PC, P.O. BOX 2903, MINNEAPOLIS, MN, 55402-0903, US

(73) Assignee: Themis Medicare Limited, Mumbai (IN)

(21) Appl. No.: 12/338,467

(22) Filed: Dec. 18, 2008

(30) Foreign Application Priority Data
Dec. 18, 2007 (IN) 2490/MUM/2007

(51) Int. Cl. C07D 309/30

(52) U.S. Cl. 549/292

(57) ABSTRACT

The present invention relates to a novel process for isolation and recovery of compounds such as biosynthetically produced simvastatin in either lactone form or in the form of its acid salt in high yield and purity, from microbial fermentation broth and isolating the said statin from harvested microbial broth.

This application claims benefit of Serial No. 2490/MUM/2007, filed Dec. 18, 2007 in India and which application is incorporated herein by reference. A claim of priority to all, to the extent appropriate is made.

### TECHNICAL FIELD OF THE INVENTION

Present invention relates to a novel process for isolation of simvastatin in lactone form or in the form of its acid salt from the fermentation broth. In particular present invention relates to isolation of simvastatin in the form of simvastatin lactone or in the form of simvastatin acid salt from the harvested fermentation broth.

### BACKGROUND AND PRIOR ART

Certain mevalonate derivatives are known to be active as anti-hyper-cholestrolemic agents, and these function by limiting cholesterol biosynthesis by inhibiting the enzyme HMG-CoA reductase. atorvastatin, lovastatin (also known as mevinolin or monacolin K), Mevastain (also known as compacting, and derivatives and analogs of these compounds such as pravastatin, simvastain are known HMG-CoA reductase inhibitors and are used as anti-hypercholesterolemic agents. Majority of them are produced by fermentation using different species belonging to Aspergillus, Monascus, Nocardia, Amycolaptosis, Mucor or Penicillium genus; others are obtained by treating the fermentation products using the method of chemical synthesis or they are produced by chemical synthesis.

Lovastatin is the first of the statin to be used widely and is manufactured by a fermentation-based process. It is produced as secondary metabolite of the fungus Aspergillus terreus (U.S. Pat. No. 4,231,938).

Mevastatin (it is compactin) and lovastatin are natural fermentation products which possess a 2-methylbutyrate side chain in the 8-position of their hexahydronaphthalene ring system. It was later on proved that products having a 2,2-dimethylbutyrate side chain in this position are more active inhibitors of HMG-CoA reductase than analogs with a 2-methylbutyrate side chain.

Lovastatin is a natural product obtained by fermentation using Aspergillus tereus possessing 2-methylbutyrate side chain in C-8 position, whereas simvastatin, its more potent analog possess 2,2-dimethylbutyrate side in C-8 position. simvastatin is produced semisynthetically from lovastatin.

Simvastatin is synthetically prepared from lovastatin in many different ways.

For example, U.S. Pat. No. 4,444,784 discloses a process in which the methyl group is introduced by using many chemical steps which ultimately result in low yield.

In U.S. Pat. No. 4,582,915 (to Merck), in one component of the process the methyl group is introduced by direct alkylation of the methylbutyrate side chain using a metal alkylamide and methylhalide. The disadvantages of this process are many, like product contamination with by-products and starting material resulting in low purity of the required product.

CA 1287063 and U.S. Pat. No. 4,820,850 (to Merck) describe a process wherein the said methylation is carried out using low boiling amines. This process also suffers from a drawback. Low boiling amines are unsafe to handle, the silyl intermediate involved in the process is oil and hence difficult to isolate after purification and characterisation, the deprotection is carried out by using hydrofluoric acid which is highly corrosive and the hydrolysis is carried out using a base thus leading to metal salt of mevilonic acid which needs an additional step of lactonisation.

Above mentioned chemical processes suffer from one or other drawbacks like use of silyl reagents or low yield of the final product.

Simvastatin exist in both the lactone as well as the hydroxyl-acid form as shown below. There is equilibrium between the two forms. Due to difference in the polarity between the two forms, isolation and subsequent purification of simvastatin is difficult. Great care has to excercised during isolation and purification of simvastatin as the method to isolate one form may not be suitable for the isolation of the other form thus there will be reduction in the overall yield.

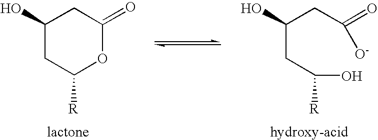

There are processes described in the literature (or prior art) for biosynthsis and isolation of statin compounds like lovastatin, compactin, and pravastatin. However no prior art reference teaches a process for industrial scale isolation and recovery of simvastatin prepared biosynthetically.

U.S. Pat. No. 5,202,029 describes a process for purification of lactone form of statin compounds by dissolving the broth in an organic solvent and eluting through HPLC column. The statin compounds which were eluted were crystallized after lengthy procedure of partial evaporation of the solvent followed by addition of water. The disadvantage of this process is that this can not be used on industrial scale as the use of column for purification of the final product make the process un economical.

U.S. Pat. No. 5,616,595 describes the technique of tangential filtration of water-insoluble compounds form the fermentation broth. This is suitable for the lactone form of the statin compounds. The drawback of this process is the need of solvents for solubilization as the compounds are water-insoluble and the multiple membranes required for filtration, and the repeated filtrations, which make the process very costly.

A process for isolation of lovastatin in the lactone form is described in U.S. Pat. No. 5,712,130. According to this patent, lovastatin is extracted from the broth by extraction with butyl acetate. Crystals of lovastatin are obtained by removal of organic phase by vacuum distillation followed by cooling. Recrystallization yields the product of 90% purity. The disadvantage of the process is the need of further purification of the product before it can be used as API, thus adding to the cost of production.

U.S. Pat. No. 6,387,258 describes the process for isolation of compactin and lovastatin from fermentated microbial broth. The process disclosed in the patent is lengthy, tedious and industrially non-viable.

Yi Tang et al. (in Appl. Enviorn. Microbilol. April 2007, p. 2054-2060) have reported synthesis of simvastatin using whole-cell biocatalysis. The method of recovering simvastain as mentioned in the publication is not suitable for industry as the process uses number of solvents, and multiple washings to remove impurities.

Hence there is need to develop a process for production of simvastatin which is simple, economical, scalable and capable of producing simvastatin in high yield and purity. Looking at the all the drawbacks, the inventors were motivated to find the process which is economical, beneficial and industrially viable using green chemistry. As the chemical processes of converting lovastatin to simvastatin are low yielding and involve the use of hazardous chemicals, it is necessary to develop an environmentally friendly process which uses the technique of fermentation for production of simvastatin. It is also desired to develop a simple, easily scalable, economical, and high yielding process for isolation of statins such as simvastatin from the said fermentation broth.

The inventors have found that simvastatin in lactone form or in the form of its acid salt can be isolated and recovered in high yield and purity, from the microbial broth without isolation of lovastatin.

Advantages of the process are, it does not require purification of lovastatin as a first step, followed by further semisynthetic procedures and use a single fermentation step to produce simvastatin. The process use the same fermentation facility as used for producing lovastatin with little modifications, materials used are relatively inexpensive and the purification steps are easily practicable.

### OBJECT OF THE INVENTION

It is an object of the invention to provide a novel process for isolation and recovery of simvastatin in lactone form or in the form of its acid salt in high yield and purity, from harvested microbial broth.

It is yet another object of the invention to provide a simple process for isolation of statin compounds such as simvastatin in lactone form or in the form of its acid salt from harvested microbial broth.

It is further object of the invention to provide an industrially scalable process for isolation of statin compounds such as simvastatin in lactone form or in the form of its acid salt from harvested microbial broth.

It is yet another object of the invention to provide an environmentally friendly process for isolation of statin compounds such as simvastatin in lactone form or in the form of its acid salt from harvested microbial broth.

It is further object of the invention to provide a cost effective process for isolation of statin compounds such as simvastatin in lactone form or in the form of an acid salt from harvested microbial broth.

### DESCRIPTION OF THE INVENTION

The present invention relates to a novel process for isolation and recovery of compounds such as biosynthetically produced simvastatin in either lactone form or in the form of its acid salt in high yield and purity, from microbial fermentation broth and isolating the said statin from harvested microbial broth.

Simvastatin is a well-known cholesterol lowering drug. It is more potent derivative of lovastatin. It is marketed by Merck Co. as Zocor. Simvastain is produced semisynthetically.

The inventors of the present invention have found that statins such as simvastatin can be produced biosynthetically using microorganisms and can be isolated in lactone form or in the form of an acid salt in high yield and purity from harvested microbial broth.

The inventors have found that simvastatin can be isolated from the fermentation broth by recovering the said statins from the mixture by various novel techniques/methods as described.

There are various advantages of the process of the present invention such as: 1) the process of the present invention does not require isolation of lovastatin in the first step; 2) simvastatin can be isolated from the fermentation broth in high yield and purity by using this process; 3) the process comprises of using simple microorganisms for producing simvastatin; 4) the methods used for isolation of simvastatin from the harvested broth are inexpensive, scalable and eco-friendly.

According an aspect of the invention there is provided a novel process for producing statin compounds such as simvastatin biosynthetically from fermentation broth and isolating the said statin in lactone form or in the form of an acid salt from harvested microbial broth. In particular there is provided a novel process for isolation of simvastatin in lactone form or in the form of an acid salt from microbial broth.

According to one aspect of the invention, there is provided a novel process for the isolation of simvastatin from microbial broth, the process comprises steps of adjusting the pH of the broth after diluting the broth or without dilution of the broth to convert the said simvastatin into the lactone form or in the acid salt form; separating the biomass containing the simvastatin compounds using standard methods of separation of solid from aqueous layer; extracting the statin/simvastatin compound from the biomass into organic solvent using standard methods of extraction; adjusting the pH of the extract to alkaline by treatment with a base to obtain the statins/simvastatin compound in the form its salt; and isolating the statins/simvastatin in the lactone form or in the form of acid salt from the mother liquor.

According to another aspect of the invention, there is provided a novel process for isolation of simvastatin from microbial broth; the process comprises the steps of adjusting the pH of the broth with an acid to pH 2-4; subjecting the broth to continuous extractor to extract the simvastatin into organic solvent; separating the organic phase; isolating the product from the organic phase by concentrating the organic phase to obtain simvastatin.

According to one more aspect of the invention, there is provided a novel process for isolation simvastatin from microbial broth comprising subjecting whole broth to multiple-stage continuous extractor where the organic solvent is introduced from one side and the broth is introduced from another side of the extractor and the two phases are discharged in the opposite directions at the end of the extraction; the simvastatin is isolated from the organic phase by using standard method of extraction and by concentrating the organic phase to obtain simvastatin.

According to yet another aspect of the invention, the process for isolation simvastatin from microbial broth comprises the steps of treating the microbial broth with a base to get pH to 9-12 separating the biomass by known techniques used for the separation of solid from water; treating the aqueous layer with an acid to pH 2-4.5; extracting with organic solvent; treating the organic layer with a base and isolating the statins as a salt; recovering the statins from the mother liquor by concentrating and crystallizing the lactone.

According to yet another aspect of the present invention there is provided a process for isolation of simvastatin from microbial broth in the form of lactone comprising the steps of: stirring the broth with an organic solvent and separating the organic layer from the biomass; concentrating the organic layer to get a concentrated mass; treating the concentrated mass with a hydrophilic solvent such as alcohol to precipitate simvastatin; filtering the precipitated simvastatin lactone. Alternatively the biomass is treated with a hydrophobic solvent such as toluene; the toluene extract is concentrated and is diluted with hexane to obtain simvastatin.

Simvastatin may be isolated in the form of lactone or in the form it's acid salt like inorganic salt or organic salt.

The organic solvent used for the extraction of the statin from the broth is selected from hydrocarbons, arylhydrocarbons, esters, carbonyl compounds such as ketones, halohydrocarbons, alcohols, ethers, either singly or in combination.

The standard method of extraction is selected from counter current method, use of continuous type extractor, use of Westfalia type extraction, use of multiple stage continuous extractor and any other method used for extraction.

The acid used for acidification is selected from inorganic acids like mineral acids, phosphoric acid perchloric acid, or organic acids such as monocarboxylic or dicarboxylic alkyl acid and monocarboxylic or dicarboxylic aryl acid.

The base used is selected from inorganic bases like alkali metal hydroxides, alkali metal carbonates, alkaline earth metal hydroxides, alkali metal carbonates, metal hydroxides, aqueous ammonia, ammonia gas, liquid ammonia or organic bases like alkyl amines, aryl amines and their amine derivatives.

Standard methods of separation of solids from water include the use of equipments like Filter press, Rotary vacuum drum filter, Solid/liquid separator, centrifuge, Decanters to separate out water and any other method of separation.

Standard method of recovering the statin from the extraction solvent include concentration of under vacuum or without applying vacuum, addition of another solvent, salting out and by adding antisolvents and crystallization techniques known by an expert skilled in the art.

Filtration is carried out by use of nutch filter, drum filter or by centrifuge.

Lactonization is generally carried out in dimethyl formamide, by addition of metallic hydrogen sulphate in the presence of charge transfer catalyst, and optionally in the presence of an anti-oxidant.

Recrystallization of simvastatin to get the product of pharmaceutical grade (purity 98%) is carried out by crystallization from solvents selected from alcohols such as methanol, and ethanol, ketones such as acetone, water and acetonitrile, preferably water and acetonitrile mixture, and optionally in the presence of an anti-oxidant.

Simvastatin broth was prepared as per the method described by Tang et al in the paper describing synthesis of simvastatin by use of whole cell biocatalyst.

Simvastatin broth has simvastatin content of at least 10 g/L of the broth.

The product was analyzed as the specifications given in EP & USP.

The invention is now illustrated by examples. The below cited examples are not limiting and can be modified suitably by the person skilled in art to get pharmacopoeial grade of simvastatin.

### EXAMPLE 1

### Isolation of Simvastain under Acidic Condition by Isolating Simvastatin as its Ammonium Salt

A fermentation broth containing 30 g/litre of simvastatin was prepared as per the method described in paper of Yi Tang et al. Broth volume of 100 KL was cooled to less than 15° C. and acidified with ortho-phosphoric acid to pH 2. The broth was taken in an extractor, allowed to stir at 25° C. and extracted with ethyl acetate till simvastatin from the broth is totally extracted into the organic layer. The organic layer was concentrated to about 50% and the organic layer was made alkaline to pH 10.8 by using ammonia gas. The basified organic layer was allowed to stand for two hours and the resulting simvastatin ammonium salt thus obtained was filtered off. The over all yield of Simvastatin ammonium salt is more than 75% and the purity of the crude Simva Ammonium salt is more than 85% ).

### EXAMPLE 2

### Isolation of Simvastain under Acidic Condition by Isolating Simvastatin as its Potassium Salt

A fermentation broth was prepared in the same way as in example 1. 1 L of the broth was acidified with hydrochloric acid to pH 3 and extracted with ethyl acetate till the broth had no simvastatin in it or till total simvastatin is extracted into the organic layer. The ethyl acetate layer is concentrated and basified to pH 8 with slurry of potassium hydroxide. The mixture was allowed to stand for 6 hours and the resulting potassium salt of simvastatin that crystallized out was filtered off. The salt was washed with chilled methanol and then dried. The potassium salt thus obtained was subjected to lactonization in dimethyl formamide, by adding metallic hydrogen sulphate, in the presence of charge transfer catalyst. The precipitation of pure simvastatin is carried out using water. The crystallisation is carried out from methanol/water in the presence of BHT (butylated hydroxyl toluene). Simvastatin of 98% purity was obtained in 90% yield.

### EXAMPLE 3

### Isolation of Simvastatin as Simvastatin Ammonium Salt by Continuous Extraction Method

1 L of fermentation broth prepared as in example 1 was cooled to 45° C. and extracted with 1 L ethyl acetate in a continuous counter current extractor cum separator while adjusting the pH of the mixture to 3.5 by adding hydrochloric acid. The ethyl acetate layer was separated and was basified to pH 10 with liquid ammonia. Simvastatin ammonium salt was crystallized in the same way as example 1. Simvastatin ammonium salt of >98% purity was obtained in nearly 85% yield.

### EXAMPLE 4

### Isolation of Simvastatin as Simvastatin Ammonim Salt by Extraction with Butyl Acetate

1 L of the filtered broth was acidified to pH 4.5 with citric acid and extracted with portions of iso-butyl acetate till simvastatin is totally extracted into the organic layer. The organic layer was concentrated and purged with ammonia to get pH 10-10.5. Ammonium salt of simvastain is isolated in the same way as example 1. The ammonium salt of simvastatin is converted to simvastatin lactone by method described in example 2. Simvastatin of >98% purity was obtained in 86% yield.

### EXAMPLE 5

### Isolation of Simvastatin using Anti Solvent

1 L of broth prepared as in example 1 was adjusted to pH2 to 5.5 by using acetic acid and extracted with methylene chloride till total simvastatin was extracted into the organic layer. The organic layer was concentrated and then cyclohexane was added to the residue till the solution became hazy. It was then allowed to cool with stirring. The precipitates thus obtained were filtered to get simvastatin which was further crystallized to get simvastatin of 98% purity in 90% yield.

### EXAMPLE 6

### Isolation of Simvastatin Lactone

1 L of the fermatation broth was mixed under agitation with 1 L water, 1 L toluene and orthophosphoric acid (20 ml of 70%) to maintain pH 3 and the mixture was stirred at about 60° C. for 10 hours. Toluene layer is distilled off under vacuum. The solution was cooled to 25° C. and n-hexane was added till the solution became hazy. The solution was allowed to stir for 5-6 hours and the precipitated simvastatin was filtered off. On recrystallization, simvastatin lactone of 98% purity was obtained in 94% yield.

### EXAMPLE 7

### Isolation of Simvastatin by Counter Current Extraction

1 L of the broth was basified to pH 9 using dilute sodium hydroxide and extracted with ethyl acetate The aqueous layer was acidified with dilute sulphuric acid to pH 4 and extracted with 500 ml ethyl acetate by using counter current method. The organic layer was separated and basified with ammonia to pH 10. Simvastain ammonium salt was filtered off and subjected to lactonization. Simvastatin was obtained in 88.7% yield with >97% purity.

### EXAMPLE 8

1 L of the filtered broth was agitated with 500 ml xylene under reflux for 10 hours. The organic layer was separated and then distilled off under vacuum. The residue was cooled to 25° C. and n-hexane (20 times) was added with stirring. The mixture was cooled to 0-5° C. and stirred for 6 hours. The crystallized simvastatin was filtered off and further purified by crystallizing from methanol in the presence of BHA (butylated hydroxyl anisole) to get simvastatin in 92.5% yield and 98% purity.

1. A process for isolation and recovery of biosynthetically produced simvastatin from microbial broth comprising the steps of:
a) adjusting the pH of the broth to acidic;
b) separating the biomass from the aqueous layer by using standard methods of separation of solid from aqueous layer;
c) extracting simvastatin from the biomass into an organic solvent;
d) adjusting the pH of the extracting solvent layer to alkaline pH;
e) isolating the crystals of simvastatin.

2. A novel process for isolation and recovery of biosynthetically produced simvastatin in either lactone form or in the form of its acid salt from microbial broth comprising the steps of:
a) adjusting the pH of the broth the basic;
b) separating the biomass from the aqueous layer by using standard methods of separation of solid from aqueous layer;
c) treating the aqueous layer with an acid to acidic pH;
d) extracting the aqueous layer with an organic solvent;
e) treating the organic layer with a base to basic pH;
f) isolating the crystals of simvastatin;

3. A process as claimed in claim 1 wherein simvastatin is isolated in the selective form of lactone form or its acid salt.

4. The process as claimed in any of claim 1 further comprising purifying simvastatin to make it pharmaceutically accepted grade.

5. A novel process for isolation and recovery of biosynthetically produced simvastatin in lactone form from microbial broth comprising the steps of:
a) providing an organic solvent to the broth;
b) separating the biomass from the layers by using standard methods of separation of solid from aqueous layer;
c) concentrating the organic layer to get a concentrated mass;
d) treating the concentrated mass with a hydrophilic solvent;
e) filtering the precipitated simvastatin lactone;
f) optinally purifying simvastatin to make it pharmaceutically acceptable grade.

6. A novel process for isolation and recovery of biosynthetically produced simvastatin in lactone form from microbial broth comprising the steps of:
a) providing an organic solvent to the broth;
b) separating the biomass from the layers by using standard methods of separation of solid from aqueous layer;
c) concentrating the organic layer to get a concentrated mass;
d) treating the concentrated mass with a hydrophobic solvent;
e) isolating simvastatin from the hydrophobic solvent by adding less polar hydrocarbon solvent;
f) optionally purifying simvastatin to make it pharmaceutically acceptable grade

7. A novel process for isolation and recovery of biosynthetically produced simvastatin in selective form of lactone and an acid salt from microbial broth comprising:
a) subjecting the whole broth to multiple stage continuous extractor wherein the broth is extracted by an organic solvent;
b) isolating simvastatin from the organic layer by concentrating the organic layer; and anti solvent is added.
c) optionally purifying simvastatin to make it pharmaceutically acceptable grade.

8. A process as claimed in claim 1 wherein the microbial broth is derived from bacterial origin or fungal origin or the origin capable of producing simvastatin.

9. A process as claimed in claim 1, wherein the organic solvent is selected from hydrocarbons, arylhydrocarbons, esters, carbonyl compounds such as ketones, halohydrocarbons, alcohols, ethers, halogenated hydrocarbons, either singly or in combination.

10. A process as claimed in claim 1 wherein acidic pH is achieved with acids selected from inorganic acids like mineral acids, phosphoric acid, perchloric acid and organic acids like alkyl carboxylic acids or aryl carboxylic acids, either singly or in combination.

11. A process as claimed in claim 1, wherein the basic pH is attained by base selected from inorganic bases like alkali metal hydroxides, alkali metal carbonates, alkaline earth metal hydroxides, alkali metal carbonates, metal hydroxides, aqueous ammonia, ammonia gas and organic bases like alkyl amines, aryl amines and their amine derivatives.

12. A process as claimed in claim 1 wherein standard methods of separation of solids from aqueous layer is selected from the use of equipments like Filter press, Rotary vacuum drum filter, Solid/liquid separator, centrifuge, Decanters to separate out water.

13. A process as claimed in claim 1, wherein extraction method is selected from direct double extraction, continuous extraction or the extraction is carried out by applying counter current principle.

14. A process as claimed in claim 1, wherein filtration is carried out by using nuch filter or drum filters or by centrifugation.

15. A process as claimed in claim 1, wherein the acidic pH range is between pH 2 and pH 6, preferably between 2.9 and 3.1.

16. A process as claimed in claim 1, wherein the basic pH range is between pH 8 and pH 12, preferably between pH 9 and pH 11, more preferably between pH 10 and pH 11.

17. A process as claimed in claim 1, wherein the extraction by the organic solvent is carried out at 20°-120° C.

18. A process as claimed in claim 1, wherein the organic solvent for extraction is in the ratio of 0.5-5 with reference to the biomass or the broth.

19. A process as claimed in claim 1 wherein the organic layer is concentrated either under reduced pressure or without reducing the pressure.

20. A process as claimed in claimed in claim 1, wherein the organic layer is concentrated at a temperature between 40°-90° C.

21. A process as claimed in claim 1, wherein the isolated yield of simvastatin is more than 80%.

22. A process as claimed in claim 1, wherein the purity of simvastatin is more than 95%, preferably more than 98%.

23. A process as claimed in claim 5 wherein the hydrophilic solvent is selected from alcohols, preferably methanol.

24. A process as claimed in claim 6, wherein the hydrophobic solvent is aromatic hydrocarbons like toluene and xylene.

25. A process as claimed in claim 5, wherein the less polar solvent is selected from hydrocarbon solvents like n-hexane, n-heptane and cyclohexane.

26. A process as claimed in claim 1 wherein the purification of simvastatin is carried out by crystallization from solvents like alcohol, ketone, water, and acetonitrile, either singly or in combination.

27. A process as claimed in claim 1, wherein the solvent for crystallization is selected from methanol, acetone, water, and acetonitrile, optionally in the presence of an antioxidant.

28. A process as claimed in claim 1, wherein the extraction is carried out at a temperature between 25° C.-150° C., preferably between 35° C.-120° C.

29. A process as claimed in claim 1, wherein the microbial broth contains at least 10 g of simvastatin per litre of the broth.
